(19) United States
(12) Patent Application Publication
(10) Pub. No.: US 2026/0272551 A1
(43) Pub. Date: Sep. 17, 2026

(54) ADAPTIVE FIBER CONTROL

(71) Applicant: Lumenis Ltd., Yokneam Ilit (IL)

(72) Inventors: Shlomi Ronen, Ranana (IL); Oded Salomon, Manof (IL)

(73) Assignee: Lumenis Ltd., Yokneam Ilit (IL)

(21) Appl. No.: 19/563,066

(22) Filed: Mar. 11, 2026

Related U.S. Application Data
(60) Provisional application No. 63/769,906, filed on Mar. 11, 2025.

(51) Int. Cl. A61B 18/26; A61B 1/00; A61B 1/018; A61B 18/00; A61B 90/00

(52) U.S. Cl. CPC A61B 18/26; A61B 1/00128; A61B 1/00133; A61B 1/018; A61B 90/08; A61B 2018/00172; A61B 2018/00184; A61B 2018/00982; A61B 2090/0807

(57) ABSTRACT

A medical apparatus includes a fiber channel having a proximal end and a distal end, a flexible bellows extending around at least the proximal end of the fiber channel, a motor, a circuitry, and a housing, the housing including a slidable component, wherein the housing surrounds at least a portion of the fiber channel, the flexible bellows, motor, and circuitry, and wherein the slidable component is located proximal of the fiber channel.

### PRIORITY

This application claims the benefit of priority under 35 USC § 119 to United States Provisional Patent Application Ser. No. 63/769,906, filed Mar. 11, 2025, which is incorporated by reference herein in its entirety and for all purposes.

### TECHNICAL FIELD

The disclosure pertains to medical devices and more particularly to a modular apparatus for advancing and retracting a laser optical fiber within a medical device.

### BACKGROUND

Medical lasers are used in a variety of procedures. Among several of the procedures, laser energy is directed towards a target using a fiber as a conduit for the laser energy. One such procedure, to address renal calculi (e.g., kidney stones) is ureteral endoscopy, or lithotripsy. An endoscopic probe, with a camera or other sensor, is inserted into the patient's urinary tract to locate the calculi for removal. In endoscopic lithotripsy, the probe also includes an optical fiber, which conducts a laser beam to disintegrate the calculi as they are found.

An ideal lithotripsy procedure is fast, precise, and thorough. The medical practitioner minimizes the time that the endoscope is inserted, directs all the discharged laser energy into target calculi, and assures that no large stones or fragments remain. However, current lithotripsy procedures require an operator to manually push and pull the endoscope to adjust the location of the fiber tip according to visual feedback.

### SUMMARY

This disclosure provides design, material, manufacturing method, and use alternatives for medical devices. An example medical apparatus comprises a fiber channel having a proximal end and a distal end, a flexible bellows extending around at least the proximal end of the fiber channel, a motor, a circuitry, and a housing. The housing comprising a slidable component, wherein the housing surrounds at least a portion of the fiber channel, the flexible bellows, motor, and circuitry, and wherein the slidable component is located proximal of the fiber channel.

Alternatively or additionally to the embodiment above, the medical apparatus is configured to couple to an endoscope.

Alternatively or additionally to any of the embodiments above, the distal end of the fiber channel is configured to be inserted into a port of the endoscope.

Alternatively or additionally to any of the embodiments above, the proximal end of the fiber channel is configured to couple to a laser fiber.

Alternatively or additionally to any of the embodiments above, the circuitry is connected to an external device, the external device configured to determine a distance measurement between a distal end of the laser fiber and a target.

Alternatively or additionally to any of the embodiments above, the external device is a laser console.

Alternatively or additionally to any of the embodiments above, the circuitry is configured to control the motor to move the laser fiber to a predetermined distance from the target.

Alternatively or additionally to any of the embodiments above, further including a visual indicator of the distance of the laser fiber tip from the target.

Alternatively or additionally to any of the embodiments above, the distal end of the fiber channel is configured to couple to a connector of a port of an endoscope to form a fluid tight seal between the port and the fiber channel.

Alternatively or additionally to any of the embodiments above, the fiber channel has a generally tapered shape.

Alternatively or additionally to any of the embodiments above, the motor is an eccentric motor or a linear motor.

Alternatively or additionally to any of the embodiments above, the housing further comprises an opening, the opening configured to receive a portion of the motor.

Alternatively or additionally to any of the embodiments above, the opening is shaped and sized to convert rotational motion of the motor into linear motion of the slidable component.

Alternatively or additionally to any of the embodiments above, further including a battery configured to power the motor

Alternatively or additionally to any of the embodiments above, the fiber channel comprises a dynamic portion and a static portion and wherein the flexible bellows couples the dynamic portion and the static portion.

An example medical apparatus includes a fiber channel having a proximal end and a distal end, the distal end of the fiber channel configured to be inserted into a port of the endoscope and the proximal end of the fiber channel configured to couple to a laser fiber, a flexible bellows extending around at least the proximal end of the fiber channel, a motor, a circuitry, a laser console, and a housing, the housing comprising a slidable component. Wherein the housing surrounds at least a portion of the fiber channel, the flexible bellows, motor, and circuitry, wherein the laser console is configured to determine a distance measurement between a distal end of the laser fiber and a target, and wherein the slidable component is located proximal of the fiber channel.

Alternatively or additionally to any of the embodiments above, the circuitry is configured to control the motor to move the laser fiber to a predetermined distance from the target.

Alternatively or additionally to any of the embodiments above, the apparatus is configured to couple to an endoscope.

Alternatively or additionally to any of the embodiments above, the motor is an eccentric motor or a linear motor.

Alternatively or additionally to any of the embodiments above, the fiber channel comprises a dynamic portion and a static portion, and wherein the flexible bellows couples the dynamic portion and the static portion.

An example endoscope includes a lumen, a port, and a connector, wherein the port is configured to couple the lumen to a laser apparatus, the laser apparatus comprising a fiber channel having a proximal end and a distal end, a flexible bellows extending around at least the proximal end of the fiber channel, a motor, a circuitry, and a housing, the housing comprising a slidable component, wherein the housing surrounds at least a portion of the fiber channel, the flexible bellows, motor, and circuitry.

Alternatively or additionally to any of the embodiments above, the proximal end of the fiber channel is configured to couple to a laser fiber.

Alternatively or additionally to any of the embodiments above, further including a laser console configured to determine a distance measurement between a distal end of the laser fiber and a target.

Alternatively or additionally to any of the embodiments above, the circuitry is configured to control the motor to move the laser fiber to a predetermined distance from the target.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a prior art endoscope;

FIG. 2 is a side view of an illustrative endoscope having an adaptive fiber control apparatus;

FIG. 3A is a side view of the endoscope of FIG. 2 including a fiber component;

FIG. 3B is a side view of the endoscope of FIG. 3A where the fiber component has been advanced by the adaptive fiber control apparatus;

FIG. 4A is an example fiber control apparatus including an eccentric motor;

FIG. 4B is an exploded view of the example fiber control apparatus of FIG. 4A;

FIG. 5 is a side view of an alternative fiber control apparatus;

FIG. 6 illustrates a block diagram of an example fiber control electrical system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures.

The following description should be read with reference to the drawings, which are not necessarily to scale. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

As introduced above, the present disclosure provides a modular apparatus configured to automatically adjust the position of a laser fiber tip during endoscopic procedures, specifically, ureteroscope procedures treating renal calculi (kidney stones). This sterile, disposable, and modular apparatus can fit to any endoscope. When treating renal calculi, the stone fragments are often “dusted” or broken up into progressively smaller and smaller fragments. This is commonly referred to as “pop-dusting: or “pop-corning.” The challenge and common question for physicians, is determining when the stone fragments are in range, or are the correct distance from the end of the fiber tip. In current ureteroscope devices, the laser fiber is locked in position relative to the ureteroscope to prevent fluid leakage, and the operator must push or pull the entire ureteroscope in order to adjust the fiber tip location.

Currently, there is no known method for automatically adjusting the distance between the laser fiber and a target. When targets are in line of site of the fiber, they will be impacted by a treatment beam and broken up. This causes the distance between the fiber tip and a target to be rapidly changing. Currently, physicians determine whether they should move the fiber based on experience, and visual feedback which can be subjective. Lack of indication of the distance can prolong lithotripsy procedures and can result in delivering excess energy to the kidneys which may heat them up with no real effect on stones. The present disclosure provides an apparatus for automatically keeping the laser fiber “in range” of a target, which can increase efficiency, and reduce procedure time and operator complexity for lithotripsy procedures.

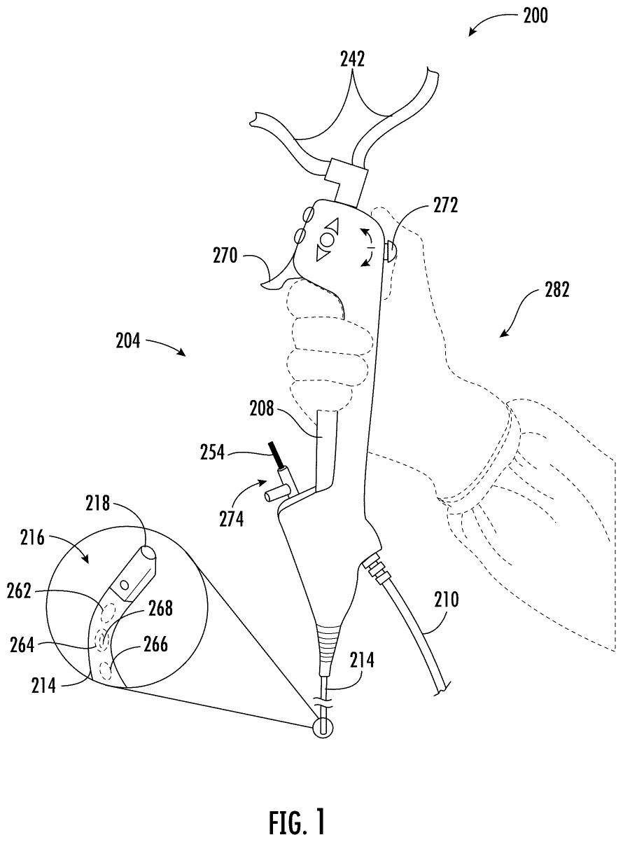

FIG. 1 illustrates a prior art ureteroscope within an operating room (OR) environment 200 which can be implemented with an endoscope such as, a ureteroscope. A physician or operator 282 holds the endoscope throughout the length of the procedure. The OR environment 200 may further include an endoscope console, operating room infrastructure, a computing system, and the like (not shown).

The endoscope can include an elongated shaft 214 coupled to endoscope handle 208, which can be used to access a patient's bladder and/or kidney. In such a procedure, the endoscope, and particularly, a distal end 216 of the elongated shaft 214 is inserted into the bladder via the urethra and can be further inserted into the kidney via the ureter, where it can be used to diagnose and/or treat a variety of problems in the urinary system. The endoscope can include a camera 218 disposed on the distal end 216 of the elongated shaft 214. The camera 218 can be used to provide a visual feed on a display screen. For example, images captured by camera 218 can be rendered and displayed on a display of an endoscope console. It is noted that the present disclosure can be applied to any of a variety of lithotripsy procedures, such as, endoscopic lithotripsy, percutaneous nephrolithotomy, or the like. It is noted that the present disclosure uses endoscopic lithotripsy as an example to describe the subject matter to be claimed. However, this is used as an example only and not intended as a limitation.

During an example lithotripsy procedure, blood and/or debris may be present in the body cavity, which may negatively affect image quality captured by the endoscope. Fluid flow (e.g., irrigation fluid flow) from a fluidics unit may be used to flush the body cavity to improve image quality. Further, as laser energy can be used to fragment, ablate, dust, or otherwise treat the stone, heat may be generated at the treatment site. Fluid flow can be used to control the temperature of the treatment site to avoid damage or injury to adjacent tissue. A fluid system may be fed through a port in the endoscope, for example a port 242 or 274. Fluid flow can be controlled with a switch 270, allowing the user 282 to control when fluid is flowing. The handle may also include other buttons 272 that perform other functions (e.g. control accessory devices).

During an example lithotripsy procedure, the treatment fiber 254 can be inserted into a port of endoscope handle 208 and pushed through a working channel (not shown) of elongated shaft 214 such that a distal end of the treatment fiber 254 can be positioned proximate to stone in urinary system. A laser generator can generate laser energy, which is optically coupled to treatment fiber 254 via an optical coupler. The laser energy is conveyed through the treatment fiber 254 and emitted from the distal end, where it may be incident on a stone to cause the stone to be treated (e.g., ablated, fragmented, dusted, etc.) In some embodiments, the endoscope may include one or more sensors, which can be disposed proximate the distal end 216 of the elongated shaft 214. Some example sensors include pressure sensor 262, temperature sensor 264, grating 266, electromagnetic sensor 268 and the like.

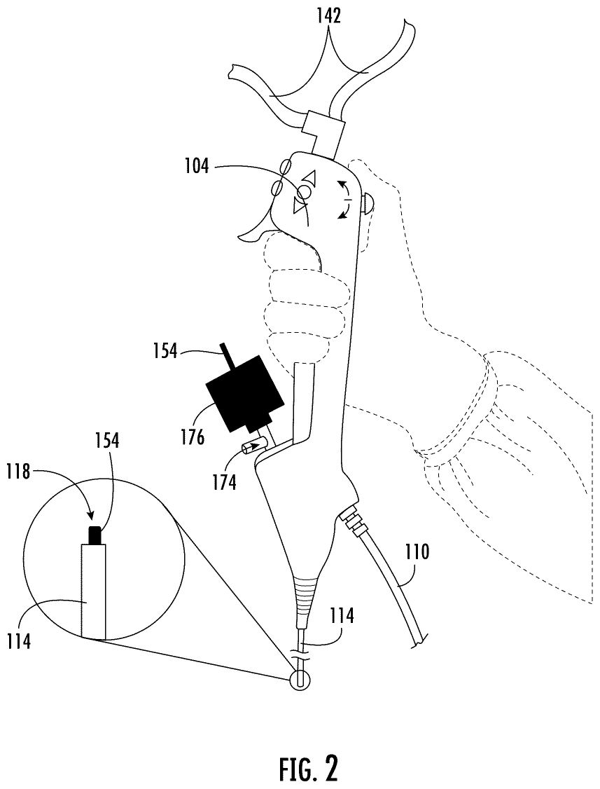

FIG. 2 illustrates an endoscope 104 as in FIG. 1, including an apparatus 176 configured to advance and retract the treatment fiber 154 through the elongate shaft 114. The treatment fiber 154 is inserted through port 174 of the endoscope 104. It should be understood that the treatment fiber 154 may be inserted into a port at any location on the endoscope 104 (e.g. ports 150, 142). As the treatment fiber 154 is advanced, the distal end 118 extends out past the elongate shaft 114 to access the bladder, kidneys, or other treatment areas. Apparatus 176 is attached to the endoscope 104, and may be releasably attached, or may be a permanent fixture. In some embodiments, the endoscope 104 may include a plug or port on an outer surface of the endoscope 104, the plug configured to receive and support the apparatus 176. Apparatus 176 is configured to receive at least a portion of the treatment fiber 154. Said differently, the apparatus is configured to attach to the endoscope, at the location where the treatment fiber is inserted (174), so that the apparatus 176 connects to both the endoscope 104 and the treatment fiber 154. In some embodiments, the apparatus 176 is configured to fluidly connect to a working channel within the endoscope 104, for example, a working channel of the elongate shaft 114, or a working channel of fluid tubing 142, 150 etc. Additionally, the endoscope 104 may be connected to a lasing system, lasing console, computing device, or other such components by the plug 110, or via wireless connection.

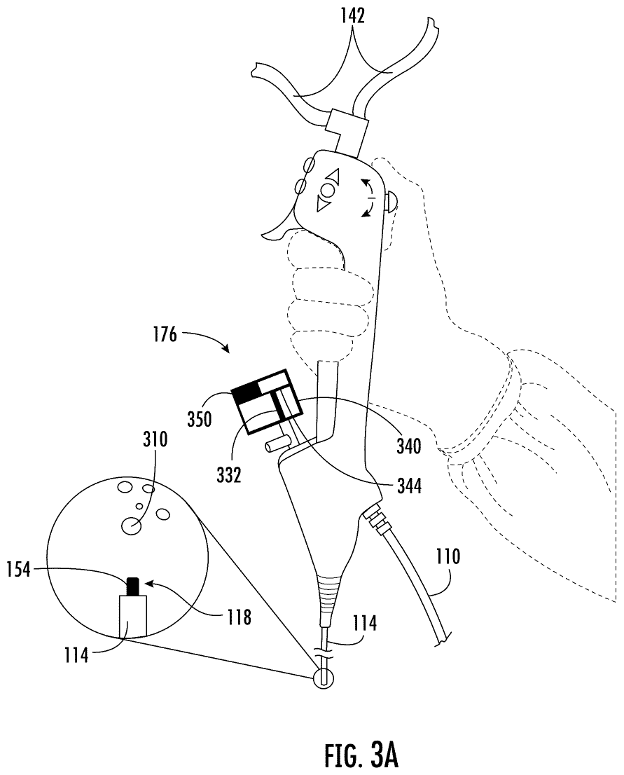

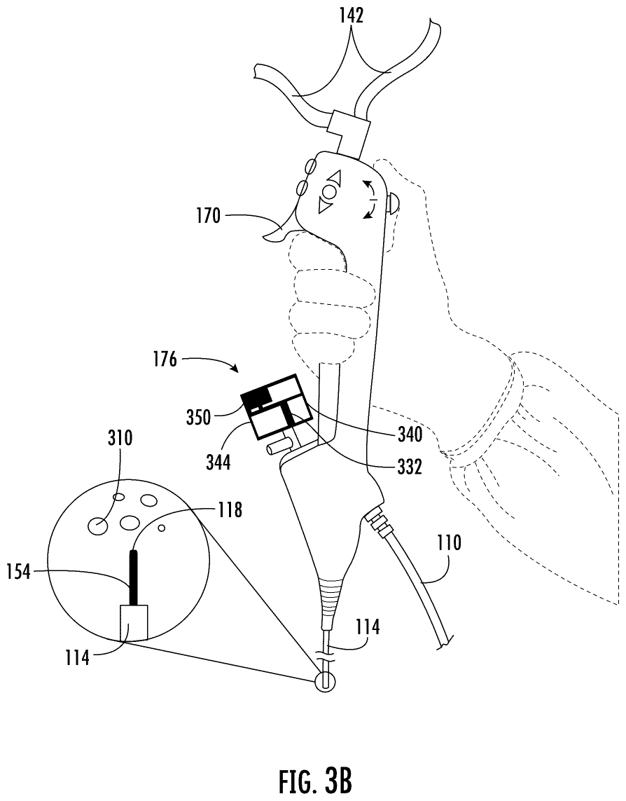

As shown in FIG. 3A, the apparatus has a motor 350 configured to control the position of the treatment fiber 154. Throughout a pop-dusting procedure, a bundle or group of stones are moved around. This movement causes the particular stone on which the treatment fiber 154 is directed to change. Thus, the distance between the distal end 118 of the treatment fiber 154 and the stone or tissue 310 is always changing. During operation, apparatus 176 can be configured to automatically adjust the position of the treatment fiber 154 relative to the stone or tissue 310. A lasing system, console, or other computing device can determine the distance of the distal end 118 of the treatment fiber 154 from a stone 310. The measured distance is then compared to a predetermined optimal distance, for example, 1 mm from a stone or tissue 310 to the fiber end 118, or 3 mm from a stone or tissue 310 to the fiber end 118, depending on the desired treatment applications. The apparatus 176 further includes a housing 340, the housing 340 containing the motor 350, plate 344, and fiber channel 332. As shown in FIG. 3B, as the motor 350 advances the plate 344 toward the distal end of the endoscope 104, the fiber channel 332 and treatment fiber 154 move toward the stones or tissues 310. Conversely, apparatus 176 can pull the treatment fiber away from the stones or tissues 310, increasing the distance between the distal end 118 and the stones or tissues 310 (FIG. 3A).

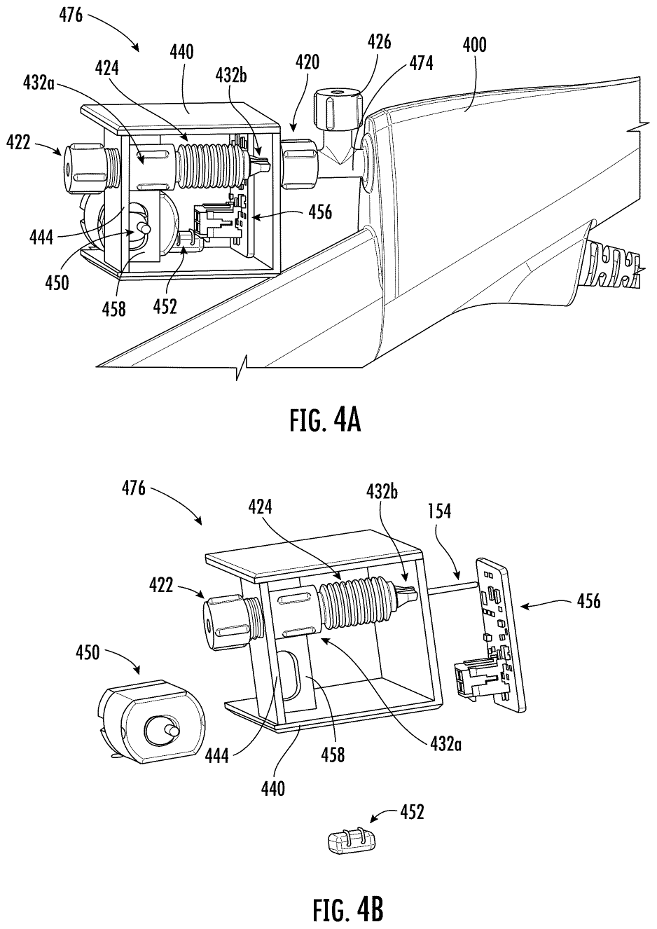

FIG. 4A illustrates an example fiber control apparatus 476. The apparatus is attached to port 474 of the endoscope 400 which contains treatment fiber 154 (FIGS. 3A-3B). It should be understood that the apparatus 467 can be shaped and sized to accommodate a variety of medical devices. The port 474 has a branch that may accommodate a fluid system, with a Tuohy-Borst 426 or other similar connector or seal configured to prevent fluid leakage. Similarly, a distal connector 420 (e.g., Tuohy-Borst, or the like) between the housing 440 and the port 474 prevents leakage from a fluid or irrigation system. A fiber channel 432 extends through the connector 420 and into the port 474 to surround the treatment fiber 154 (FIG. 3A).

In general, the fiber channel comprises a dynamic portion 432a and a static portion 432b. The fiber channel 432 may have an area of reduced diameter or a generally tapered shape (e.g., at the static portion 432b, or the like) to form a seal around the treatment fiber 154. In some embodiments, the dynamic portion 432a of the fiber channel 432 may include a clamp and/or teeth, to form a secure connection with the treatment fiber 154. Said differently, the dynamic portion 432a of the fiber channel 432 is fixed in position relative to the treatment fiber 154 and forms a tight seal between the port 474 and the apparatus 476. In some embodiments the dynamic portion 432a of the fiber channel 432 can be locked or unlocked relative to the treatment fiber 154. Current lithotripsy devices require that the treatment fiber 154 is locked in position to prevent fluid leakage from the endoscope. The static portion 432b of the fiber channel 432 surrounds the treatment fiber 154, allowing the treatment fiber 154 to move freely within the static portion 432a of the fiber channel 432 and the endoscope 400, without fluid leaking out of the port 474.

Fiber control apparatus 476 further includes a proximal Tuohy-Borst 422. With some embodiments, the proximal Tuohy-Borst 422 is configured to prevent relative motion between the fiber 154 and the dynamic portion 432a and also serve as a watertight seal while the fiber 154 moves freely inside the static portion 432b of the fiber channel 432. With other embodiments, the distal connector 420 (or Tuohy-Borst) is semi-locked directly on the fiber 154 creating a watertight seal while allowing the fiber 154 to move freely inside it. In such embodiments, the housing 440 can be mounted on the port 474 (or endoscope 400) without the need for bellows 424 since the fluid is maintained on the distal side of the distal connector 420 (Tuohy-Borst) by the watertight seal.

An eccentric motor 450 within the housing 440, is coupled to a slidable plate 444. At least a portion of the motor 450 fits and moves within an opening 458 to translate rotational motion from the motor 450 into linear movement of the plate 444. For example, as the motor 450 spins clockwise within the opening 458, the plate 444 moves distally. The plate 444 is coupled to a flexible bellow 424, which connects to the fiber channel 432. Bellow 424 provides a flexible seal between the plate 444 and the dynamic potion 432a and static portion 432b of the fiber channel 432, allowing for linear motion while preventing fluid from leaking out from the endoscope. As the plate 444 moves, the flexible bellows 424 expands and contracts to maintain a sealed environment. The plate 444 is also connected to the dynamic portion 432a of the fiber channel 432, such that linear motion of the plate 444 corresponds to linear motion of the dynamic portion 432a of the fiber channel 432. Said differently, as the motor 450 pushes and pulls the plate 444, the plate 444 pushes and pulls on the dynamic portion 432a of the fiber channel 432 and thus the treatment fiber 154. A proximal Tuohy-Borst 422, or other similar connector or seal, is disposed on the proximal side of the plate 444, effectively coupling the plate 444 to the fiber channel 432 (e.g., at the dynamic portion 432a of the fiber channel 432) and bellows 424. In some embodiments, the provides a tight seal. With specific embodiments, the tight seal can be watertight. In some embodiments, the seal can be watertight but still provide for some movement between the fiber 154 and fiber control apparatus 476.

The motor 450 may be configured to automatically advance and retract the treatment fiber 154. A circuitry 456 is disposed within the housing and is configured to control the movement of the motor based on information obtained from an outside source (e.g. laser console, camera, computing system, visual feedback, or the like). The circuitry 456 may be connected to one or more outside sourced by a wireless connection (e.g. Bluetooth) or by wired connection (e.g. USB). A battery 452 provides power to the apparatus 476. It should be understood that there may be a plug and/or cord configured to couple the apparatus 476 to an external power source. As discussed in more detail above with respect to FIGS. 3A-3B, a lithotripsy procedure requires that the treatment fiber 154 be a desired distance from a treatment area or target (e.g. stones 310, tissue, etc.) for optimal procedure efficiency. The circuitry can be configured to automatically adjust the position of the distal end 118 of the treatment fiber 154 (FIG. 3A) based on measured distance information received from an external source. This automatic adjustment keeps the distal end 118 of the treatment fiber 154 a predetermined distance away from a treatment location.

FIG. 4B shows an exploded view of the modular apparatus 476 of FIG. 4A. The housing 440 contains the flexible bellows 424, which enables linear motion of the treatment fiber 154. Further, the flexible bellows may provide a fluid tight seal around the fiber channel 432. The housing 440 has a precut opening 458 configured to receive a portion of the eccentric motor 450, the opening 458 configured to convert rotational motion of the motor 450 into linear motion of the plate 444. The circuitry 456 and battery 452 are coupled to the housing

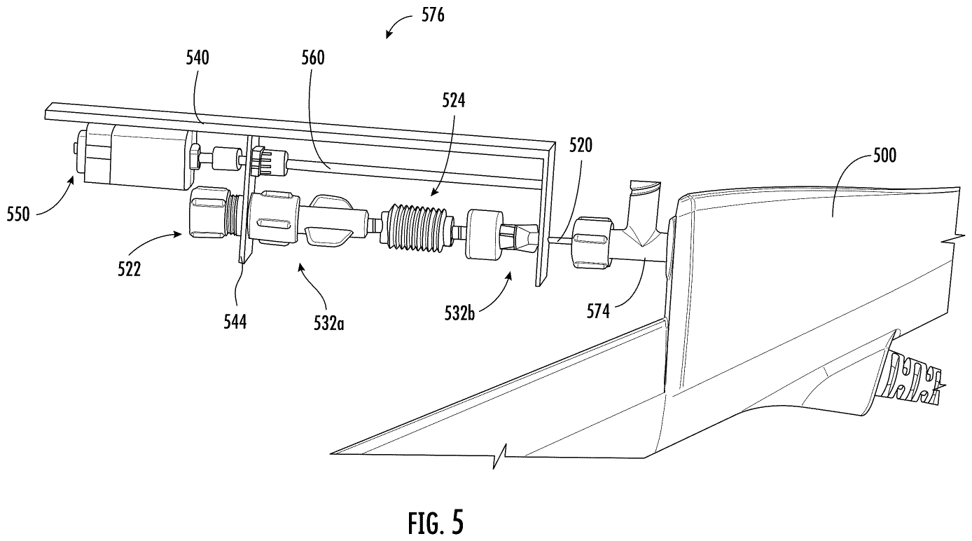

FIG. 5 illustrates an alternative modular apparatus 576 having a linear motor 550. The motor 550 is coupled to a lead screw 560 and is configured to advance and retract the plate 544. The plate 544 is coupled to a fiber channel 532 and flexible bellows 524, and the dynamic portion 532a of the fiber channel 532 moves in response to the plate 544. For example, as the plate 544 is advanced, the dynamic portion 532a of the fiber channel 532 is advanced. As the plate 544 is retracted, the dynamic portion 532a of the fiber channel 532 is retracted. The fiber channel 532 extends through the port 574 of the endoscope 500 to surround the treatment fiber 154 (FIG. 3A). Movement of the dynamic portion 532a of the fiber channel 532 corresponds to movement of the treatment fiber 154. The apparatus 576 is attached to port 574 of the endoscope 500 which contains treatment fiber 154 (FIGS. 3A-3B). It should be understood that the apparatus 576 can be shaped and sized to accommodate a variety of medical devices and endoscopes. A distal end 520 of the fiber channel 532 can be coupled to the port 574 (e.g., via a connector, or the like). Additionally, a proximal connector 522 is disposed around a proximal end of the fiber channel 532 to create a sealed environment within the fiber channel 532. The flexible bellows 524 coupled the dynamic portion 532a and the static portion 532b of the fiber channel 532 to allow for linear movement without breaking the seal, thereby preventing fluid from leaking out of a fluid or irrigation system within the endoscope 500.

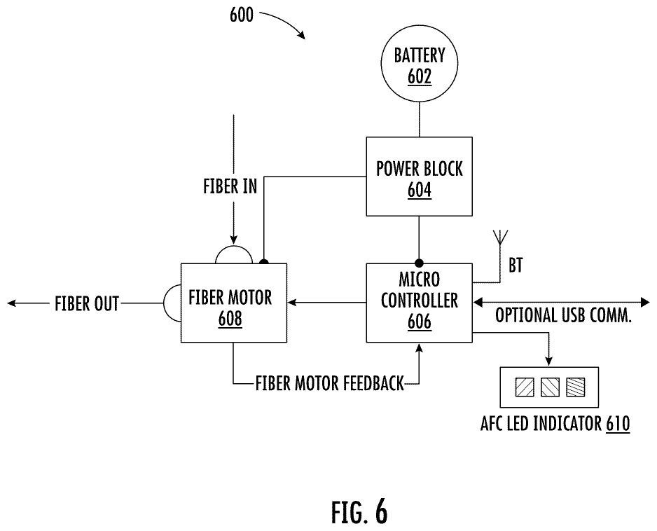

FIG. 6 illustrates a block diagram of an electrical system 600 of an adaptive fiber control apparatus as disclosed herein. The electrical system 600 includes a battery 602 configured to supply power to a power block 604. The power block 604 is configured to hold power from the battery 602 and distribute it between other systems within the electrical system 600. For example, the power block 604 directs power to the motor 608, to control movement of a fiber forwards and backwards. The power block 604 is further configured to direct power toward a micro controller 606. The micro controller 606 may execute instructions from an external input device (e.g. processor, laser console etc.). The micro controller 606 may be in communication with external input devices via coaxial components, USB, Bluetooth connection, audio, analog, stereo, radio frequency, high-definition multimedia interface (HDMI) or the like. The micro controller 606 communicates with fiber motor 608, to drive the motor, in turn moving the fiber.

The fiber motor 608 is in communication with the micro controller 606, to form a feedback loop, allowing the micro controller 606 to automatically adjust the position of the fiber based on information received from the motor 608 and information received from an external source. Measured distance from an external source can be used as feedback to the micro controller 606 to maintain an optimal distance between a fiber and a target. A visual indicator 610 may be connected to the electrical system 600. The visual indicator configured to dynamically display an indication of distance between a laser fiber and a target, or to display if the fiber is within a certain distance from a target, or a “threshold distance.” The visual indicator 610 may be connected to the micro controller 606, and/or an external device. In some embodiments, the visual indicator 610 is located on an external device. The visual indicator may include a colored bar, colored lights, words, charts, or graphical indication such as a pattern. To indicate to a user a distance measurement or threshold. It is noted that although a visual indicator is depicted in this figure and described above, any of a variety of indicators could be implemented, such as, for example, a sound indicator, a haptic feedback indicator, or the like.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical apparatus comprising:
a slidable component to couple to an optical fiber;
a motor coupled to and configured to actuate the slidable component; and
a circuitry coupled to the motor,
wherein the circuitry is configured to cause the motor the move the optical fiber via the slidable component.

2. The medical apparatus of claim 1, wherein the apparatus is configured to couple to an endoscope.

3. The medical apparatus of claim 2, further comprising a fiber channel, wherein a distal end of the fiber channel is configured to be inserted into a port of the endoscope, and wherein the optical fiber is to be inserted into a proximal end of the fiber channel.

4. The medical device of claim 1, wherein the circuitry is connected to an external device, the external device configured to determine a distance measurement between a distal end of the optical fiber and a target.

5. The medical apparatus of claim 1, wherein the optical fiber is configured to couple to a connector of a port of an endoscope to form a fluid tight seal between the port and the optical fiber.

6. The medical device of claim 5, wherein the external device is a laser console.

7. The medical device of claim 5, wherein the circuitry is configured to control the motor to move the laser fiber to a predetermined distance from the target.

8. The medical system of claim 5, further comprising a visual indicator of the distance of the laser fiber tip from the target.

9. The medical apparatus of claim 3, wherein the distal end of the fiber channel is configured to couple to a connector of a port of an endoscope to form a fluid tight seal between the port and the fiber channel.

10. The medical apparatus of claim 1, comprising a flexible bellows extending around at least the proximal end of the fiber channel.

11. The medical apparatus of claim 1, wherein the motor is an eccentric motor or a linear motor.

12. A medical apparatus comprising:
a fiber channel having a proximal end and a distal end, the distal end of the fiber channel configured to be inserted into a port of the endoscope and the proximal end of the fiber channel configured to couple to a laser fiber;
a flexible bellows extending around at least the proximal end of the fiber channel;
a motor;
a circuitry;
a laser console; and
a housing, the housing comprising:
a slidable component;
wherein the housing surrounds at least a portion of the fiber channel, the flexible bellows, motor, and circuitry,
wherein the laser console is configured to determine a distance measurement between a distal end of the laser fiber and a target, and
wherein the slidable component is located proximal of the fiber channel.

13. The medical apparatus of claim 12, wherein the circuitry is configured to control the motor to move the laser fiber to a predetermined distance from the target.

14. The medical apparatus of claim 12, wherein the apparatus is configured to couple to an endoscope.

15. The medical apparats of claim 12, wherein the motor is an eccentric motor or a linear motor.

16. The medical apparatus of claim 15, wherein the fiber channel comprises a dynamic portion and a static portion, and wherein the flexible bellows couples the dynamic portion and the static portion.

17. An endoscope comprising:
a lumen;
a port; and
a connector;
wherein the port is configured to couple the lumen to a laser apparatus, the laser apparatus comprising:
a fiber channel having a proximal end and a distal end;
a flexible bellows extending around at least the proximal end of the fiber channel;
a motor;
a circuitry; and
a housing, the housing comprising:
a slidable component;
wherein the housing surrounds at least a portion of the fiber channel, the flexible bellows, motor, and circuitry.

18. The endoscope of claim 17, wherein the proximal end of the fiber channel is configured to couple to a laser fiber.

19. The endoscope of claim 18, further comprising a laser console configured to determine a distance measurement between a distal end of the laser fiber and a target.

20. The endoscope of claim 18, wherein the circuitry is configured to control the motor to move the laser fiber to a predetermined distance from the target.
